SEQUENCE LISTING (2 sequences)

SEQ ID NO: 1 - DNA, 4257 bp, synthetic construct
  source 1..4257: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgattaatgcgtctcggttttagagctagaaatagcaagttaaaataaggctagtccgttatcaacttgaaaaagtggcaccgagtcggtgctttttttataacttcgtatagcatacattatacgaagttatggcgcgccactggatccgctcgtacgtagaagcttccgccgcagtctcacgcccggagcgtagcgaccgagtgagctagctatttgtttatttttctaaatacattcaaatatgtatccgctcatgagacaataaccctgataaatgcttcaataatattgaaaaaggaagagtatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccttggcgttatccagctaagcgcgaactgcaatttggagaatggcagcgcaatgacattcttgcgggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaataacttcgtatagcatacattatacgaagttatnnnnnnnngaataaaacgcacgggtgttgaattcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 7267 bp, synthetic construct
  source 1..7267: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttgcagatctgcgactctagaggatctgcgactctagaggatcataatcagccataccacattttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccttaattaagtgtctagaggttccaccctcatgttggttgatcttcaacattgtcctgacttcttcccacttgacattcctcagggtcctgtgatcatggctgggtctagtgaggttcaaacctgcactgccctacccacacccacacccagctcagcgtcagtcaggatcaacagttacctagagatcatctttctggggcttaagcattggtgggagcagatgggatatgagttggggatttggggatgggggaagatatctgctccccctccccctacaccctagccttttaaaaggccttctcaggtcagagaccaggagaaaagtataggagagatacacaatggaccaggaagaagaaaagggagagggaggctcagaccttctagacaaggtaagagggctctggctgactccaccatccgcttcttgaggtctcggcacctgtaattgacaagattaattcatttatagggcatctaattagcaagtaagtctctggagtcccctgacccagttactataacacacagggggtataggtaggggagtataagagcccctcctcagggcaaatgaatggattcttagtactgtcccccaagagatagtaggtactaggatttaggggcacttctgagccccatttccctggtaagtgtcccaaccccccaaatcaacccaagcctggtctcaatctaggacagtggtagaatgctgtccctagagtcagtaccatgtgaaattgtgctgcaggcaggggccccaggctgggaggtgggggttgggggagtcaggggcaggtcagggaaggagactcaggtttcatttagagaaattctgcagacccgtgaggactggatccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacatagtgatgaactacatcagaaacctggactctgagactggggccatggtgaggctgctcgaggatggggacggaagcggagccactaacttctccctgttgaaacaagcaggggatgtcgaagagaatcccgggccaatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtccggaggaggacccaagaagaagaggaaggtgtagtaaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttacgccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2025/0295813 A1
(43) Pub. Date: Sep. 25, 2025

(54) VIRAL VECTORS FOR INCREASING THE SPECIFICITY OF GENE EXPRESSION

(71) Applicant: The Trustees of Columbia University in the City of New York, New York, NY (US)

(72) Inventors: Lai Wei, New York, NY (US); Alejandro Chavez, New York, NY (US)

(21) Appl. No.: 19/229,699

(22) Filed: Jun. 5, 2025

Related U.S. Application Data
(63) Continuation of application No. PCT/US2023/082573, filed on Dec. 5, 2023.
(60) Provisional application No. 63/386,106, filed on Dec. 5, 2022.

(51) Int. Cl. A61K 48/00; C12N 9/22; C12N 15/11; C12N 15/86

(52) U.S. Cl. CPC A61K 48/0058; C12N 9/226; C12N 15/11; C12N 15/86; C12N 2310/20; C12N 2750/14143

(57) ABSTRACT

The present disclosure provides viral vectors which decrease undesired or off-target gene expression. Particularly, the present disclosure provides recombinant viral vectors comprising a transcription termination sequence adjacent to an inverted terminal repeat sequence (e.g., downstream of a first inverted terminal repeat sequence and upstream of an expression cassette), and virus or virus-like particles, compositions, and methods of using thereof.

### FIELD

The present disclosure relates to viral vectors which decrease undesired or off-target gene expression. Particularly, the present disclosure relates to recombinant viral vectors comprising a transcription termination sequence adjacent to an inverted terminal repeat sequence (e.g., downstream of a first inverted terminal repeat sequence and upstream of an expression cassette), and virus or virus-like particles, compositions, and methods of using thereof.

### CROSS-REFERENCE TO RELATED APPLICATIONS

This application is a continuation of PCT International Application No. PCT/US2023/082573 filed Dec. 5, 2023, which claims the benefit of U.S. Provisional Application No. 63/386,106 filed Dec. 5, 2022, the contents of which are herein incorporated by reference in its entirety.

### SEQUENCE LISTING STATEMENT

The content of the electronic sequence listing titled COLUM-41432.601.xml (Size: 14,335 bytes; and Date of Creation: Dec. 5, 2023) is herein incorporated by reference in its entirety.

### BACKGROUND

Gene therapy and genetic engineering largely rely on the ability to introduce genetic material into cells to alter gene and/or protein expression. Adeno-associated viruses (AAVs) and vectors thereof are the safest and most effective delivery vehicles to deliver genetic material into a broad range of cell types. AAVs are small, non-enveloped viruses with a single-stranded 4.7 kb DNA genome flanked by 2 Inverted Terminal Repeats (ITRs). There are 3 genes within the viral genome: Rep (Replication) is responsible for viral replication and packaging, Cap (Capsid) encodes 60 outer coat proteins that protect the genomic DNA and direct cell binding, and Aap (Assembly activating protein) provides a scaffold for capsid assembly. In order to make AAVs more suitable for a broader range of applications, recombinant AAVs (rAAVs) have been engineered to have most of the viral genome replaced with sequences containing a promoter, genes of interest, and a terminator, leaving only the ITRs. Engineered viral vectors, and virus particles comprising the engineered vectors, offer improved transduction properties, specificity, dosages, and/or immunoreactivity, thereby adding to the available tools for gene therapy and genetic engineering.

### SUMMARY

Provided herein are recombinant or engineered viral vectors that facilitate control of expression of a desired gene product.

In some embodiments, the recombinant or engineered viral vectors comprise a transcription termination sequence adjacent, either upstream or downstream, to a first inverted terminal repeat (ITR) sequence, a transcription termination sequence adjacent, either upstream or downstream, to a second inverted terminal repeat (ITR) sequence, or a combination thereof; and an expression cassette flanked by at least one of the first and second ITR sequences.

In some embodiments, the transcription termination sequence is immediately adjacent to its respective ITR sequence. In some embodiments, the transcription termination sequence is separated from its respective ITR sequence by one or nucleotides.

In some embodiments, the recombinant or engineered viral vectors comprise a first transcription termination sequence adjacent, either upstream or downstream, to a first ITR sequence and an expression cassette downstream of the transcription termination sequence and the ITR sequence.

In some embodiments, the recombinant or engineered viral vectors comprise a second transcription termination sequence adjacent, either upstream or downstream, to a second ITR sequence and an expression cassette upstream of the transcription termination sequence and the ITR sequence.

In some embodiments, the recombinant or engineered viral vectors comprise a transcription termination sequence downstream of a first ITR sequence and upstream of an expression cassette. In some embodiments, the recombinant or engineered viral vectors further comprise a second ITR sequence downstream of the expression cassette.

In some embodiments, the recombinant or engineered viral vectors comprise from 5′ to 3′: a first inverted terminal repeat sequence, a transcription termination sequence, an expression cassette, and a second inverted terminal repeat sequence. In some embodiments, the recombinant or engineered viral vectors comprise from 5′ to 3′: a transcription termination sequence, a first inverted terminal repeat sequence, an expression cassette, and a second inverted terminal repeat sequence.

In some embodiments, the recombinant or engineered viral vectors comprise from 5′ to 3′: a first inverted terminal repeat sequence, an expression cassette, a transcription termination sequence, and a second inverted terminal repeat sequence. In some embodiments, the recombinant or engineered viral vectors comprise from 5′ to 3′: a first inverted terminal repeat sequence, an expression cassette, a second inverted terminal repeat sequence, and a transcription termination sequence.

In some embodiments, the recombinant or engineered viral vectors comprise from 5′ to 3′: a first inverted terminal repeat sequence, a first transcription termination sequence, an expression cassette, a second transcription termination sequence, and a second inverted terminal repeat sequence. In some embodiments, the recombinant or engineered viral vectors comprise from 5′ to 3′: a first transcription termination sequence, a first inverted terminal repeat sequence, an expression cassette, a second transcription termination sequence, and a second inverted terminal repeat sequence.

In some embodiments, the recombinant or engineered viral vectors comprise from 5′ to 3′: a first inverted terminal repeat sequence, a first transcription termination sequence, an expression cassette, a second inverted terminal repeat sequence, and a second transcription termination sequence. In some embodiments, the recombinant or engineered viral vectors comprise from 5′ to 3′: a first transcription termination sequence, a first inverted terminal repeat sequence, an expression cassette, a second inverted terminal repeat sequence, and a second transcription termination sequence.

In some embodiments, the first inverted terminal repeat sequence and/or the second inverted terminal repeat sequence are wild-type sequences. In some embodiments, the first inverted terminal repeat sequence and/or the second inverted terminal repeat sequence are engineered ITR sequences or variant ITR sequences comprising one or more nucleotide substitutions, additions, or deletions compared to a wild-type sequence.

In some embodiments, the recombinant or engineered viral vectors further comprise a polyadenylation (Poly(A)) signal sequence downstream of the expression cassette. In some embodiments, the recombinant or engineered viral vectors comprise from 5′ to 3′: a first inverted terminal repeat sequence, a transcription termination sequence, an expression cassette, a Poly(A) signal sequence, and a second inverted terminal repeat sequence.

In some embodiments, the transcription termination sequence comprises one or more copies of a Poly(A) signal sequence. In some embodiments, the transcription termination sequence comprises two or more (e.g., two, three, four, five, six, seven, eight, nine, ten, or more) copies of a Poly(A) signal sequence.

In some embodiments, the expression cassette comprises one or more regulatory control elements. In some embodiments, the one or more regulatory control elements comprises a promoter

In some embodiments, the expression cassette comprises a heterologous sequence encoding one or more gene products of interest operably coupled to the one or more regulatory control elements. In some embodiments, the one or more gene products comprise one or more proteins, one or more RNAs, or a combination thereof.

In some embodiments, the recombinant or engineered viral vectors comprise a single strand of DNA. In some embodiments, the recombinant or engineered viral vectors are derived from a virus in the Parvoviridae family. In some embodiments, the recombinant or engineered viral vectors are derived from an adeno-associated virus.

Further provided are virus or virus-like particles comprising a recombinant or engineered viral vector described herein.

Also provided are compositions comprising a recombinant or engineered viral vector described herein. In some embodiments, the compositions further comprise a pharmaceutically acceptable carrier.

Additionally provided are methods for delivering a nucleic acid to a cell. The methods comprise contacting the cell with an effective amount of a recombinant or engineered viral vector, a virus or virus-like particle, or a composition as described herein.

In some embodiments, the expression cassette encodes a system for genetic engineering. In some embodiments, the system for genetic engineering encodes at least one components of a CRISPR-Cas system. In some embodiments, the expression cassette encodes a therapeutic or prophylactic gene product. In some embodiments, the expression cassette comprises a sequence having homology with a genomic nucleic acid in the cell.

In some embodiments, the cell is in a subject. In some embodiments, the method comprises administering the recombinant viral vector, the virus or virus-like particle, or the composition to the subject.

Other aspects and embodiments of the disclosure will be apparent in light of the following detailed description.

### BRIEF DESCRIPTION OF THE DRAWINGS

FIGS. 1A and 1B show an exemplary strategy to express Cas9 protein specifically in Purkinje cells of a mouse model. The Rosa26LSL-Cas9 Cas9 mouse model contains Cas9 and cGFP gene downstream of a Lox-Stop-Lox (LSL) transcriptional termination cassette. In this mouse model, Cas9 and eGFP is only expressed upon CRE-mediated excision of the LSL sequence (FIG. 1A). The CRE recombinase can be efficiently packaged along with the required regulatory sequences into Adeno Associated Virus (AAV) made with the PHP.cB serotype, which is able to readily infect Purkinje cells when injected intravenously (FIG. 1B).

FIG. 2 are images showing the non-specific expression of Cas9 and eGFP induced by intravenously injection of AAV-PHP.cB-L7-6-Cre. A large amount of non-specific CRE activity, indicated by expression of eGFP, was observed in numerous cells throughout the brain including the cortex, hippocampus, thalamus, brain stem, and various non-Purkinje cells within the cerebellum.

FIGS. 3A and 3B show the structure of AAV vector for highly specific expression of CRE in Purkinje cells. As shown in FIG. 3A, the expression of CRE in the AAV vector is driven by two promoters. When AAV-PHP.cB infects Purkinje cells, the L7-6 promoter will be turned on and lead to robust CRE expression. In this situation, the small amount of transcriptional activity from the ITR element will play a minimal role in controlling CRE expression in Purkinje cells. However, when AAV-PHP.eB infects a non-Purkinje cell the L7-6 promoter is turned off, yet the leaky promoter activity from the ITR element enables CRE to be expressed (albeit at lower levels than when driven by the L7-6 promoter) causing the excision of the LSL element upstream of Cas9 to still occur. As shown in FIG. 3B, the transcriptional termination signal 3×Poly(A) was inserted between the ITR sequence and the L7-6 promoter to block the transcriptional activity from the ITR element. In addition, a nuclei localization sequence (NLS) is also attached to CRE to further prevent its potential leakage to surrounding neurons via released vesicles from CRE expressing neurons. An NLS tagged fluorescence protein tdTomato was also linked to CRE recombinase by a 2A self-cleaving peptides (P2A) to indicate the expression level of CRE and for further single nuclei isolation.

FIG. 4 is images showing the non-specific CRE activity was significantly reduced after insertion of 3×PolyA between ITR and L7-6. The ITR mediated transcription of CRE in non-Purkinje cells was blocked by 3×Poly(A) inserted between ITR and L7-6, which greatly reduced the inappropriate CRE expression from most of non-Purkinje cells.

FIG. 5 is representative zoomed-in microscope images of cerebellum, hippocampus, and cortex showing the significantly reduced non-specific CRE activity. Higher magnification of microscope images of different areas in the mouse brain shows significantly reduce non-specific CRE activity.

### DETAILED DESCRIPTION

Provided herein are viral vectors which lessen or prevent inappropriate gene expression, e.g., off-target gene expression. The vectors comprise a transcriptional termination sequence prior to the regulatory elements (e.g., promoter) operably linked to a sequence encoding the gene product(s) of interest. Inclusion of the transcriptional termination sequence in such an orientation reduces unwanted gene expression, particularly for those gene products under control of non-constitutive regulatory elements (e.g., inducible, tissue/cell specific, etc.). These vectors facilitate increased dosages, which is particularly important for those vectors and expression cassettes which would otherwise result in toxicity due to inappropriate gene expression, and increased effectiveness due to a reduction in off-target expression.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV218, AAV2g9, AAV-LK03, AAV7m8, AAV Anc80, AAV PHP.B, and any other AAV including chimeric AAV. Sec, e.g., BERNARD N. FIELDS et al., VIROLOGY, volume 2, chapter 69 (4th ed., Lippincott-Raven Publishers). A number of AAV serotypes and clades have been identified (see, e.g., Gao et al, (2004) J. Virology 78:6381-6388; Moris et al, (2004) Virology 33: 375-383). “Adeno-associated virus” or AAV also encompasses chimeric AAV. The term “chimeric AAV” refers to an AAV comprising a protein capsid comprising capsid protein subunits with regions, domains, individual amino acids that are derived from two or more different serotypes of AAV or another virus, including for example, another parvovirus.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215 (3): 403-410 (1990), Beigert et al., Proc. Natl. Acad. Sci. USA, 106 (10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21 (7): 951-960 (2005), Altschul et al., Nucleic Acids Res., 25 (17): 3389-3402 (1997), and Gusfield, Algorithms on Strings, Trees and Sequences, Cambridge University Press, Cambridge UK (1997)).

The term “homology” and “homologous” refers to a degree of identity. There may be partial homology or complete homology. A partially homologous sequence is one that is less than 100% identical to another sequence.

A “vector” or “expression vector” is a replicon, such as plasmid, phage, virus, or cosmid, to which another DNA segment may be attached or incorporated so as to bring about the replication, transcription, or expression of the attached segment in a cell.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement of the viral vectors, virus or virus-like particles, or compositions of the disclosure into a cell, organism, or subject by a method or route which results in at least partial localization of the viral vectors, virus or virus-like particles, or compositions to a desired site. The viral vectors, virus or virus-like particles, or compositions can be administered by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Viral Vectors

Disclosed herein are viral vectors useful for decreasing the amount of unwanted gene expression and uncontrolled gene expression. The viral vectors comprise a first transcription termination sequence adjacent, either upstream (5′) or downstream (3′), to a first inverted terminal repeat (ITR) sequence and/or a second transcription termination sequence adjacent, either upstream (5′) or downstream (3′), to a second inverted terminal repeat (ITR) sequence; and an expression cassette flanked by at least one of the first and second ITR sequences.

In some embodiments, the transcription termination sequence is immediately adjacent to its respective ITR sequence. In some embodiments, the transcription termination sequence is separated from its respective ITR sequence by one or more (e.g., 1, 2, 3, 4, 5, 6, 7, 8, 9, 10, 15, 20, or more) nucleotides.

For example, the recombinant or engineered viral vectors comprise a first transcription termination sequence adjacent, either upstream or downstream, to a first ITR sequence and an expression cassette downstream of the transcription termination sequence and the ITR sequence; or a second transcription termination sequence adjacent, either upstream or downstream, to a second ITR sequence and an expression cassette upstream of the transcription termination sequence and the ITR sequence; or a first transcription termination sequence adjacent, either upstream or downstream, to a first ITR sequence and a second transcription termination sequence adjacent, either upstream or downstream, to a second ITR sequence and an expression cassette flanked upstream by the first ITR sequence and the first transcription termination sequence and downstream by the second ITR sequence and the second transcription termination sequence.

In some embodiments, the recombinant or engineered viral vectors comprise from 5′ to 3′: a first inverted terminal repeat sequence, a transcription termination sequence, an expression cassette, and a second inverted terminal repeat sequence. In some embodiments, the recombinant or engineered viral vectors comprise from 5′ to 3′: a transcription termination sequence, a first inverted terminal repeat sequence, an expression cassette, and a second inverted terminal repeat sequence.

In some embodiments, the recombinant or engineered viral vectors comprise from 5′ to 3′: a first inverted terminal repeat sequence, an expression cassette, a transcription termination sequence, and a second inverted terminal repeat sequence. In some embodiments, the recombinant or engineered viral vectors comprise from 5′ to 3′: a first inverted terminal repeat sequence, an expression cassette, a second inverted terminal repeat sequence, and a transcription termination sequence.

In some embodiments, the recombinant or engineered viral vectors comprise from 5′ to 3′: a first inverted terminal repeat sequence, a first transcription termination sequence, an expression cassette, a second transcription termination sequence, and a second inverted terminal repeat sequence. In some embodiments, the recombinant or engineered viral vectors comprise from 5′ to 3′: a first transcription termination sequence, a first inverted terminal repeat sequence, an expression cassette, a second transcription termination sequence, and a second inverted terminal repeat sequence.

In some embodiments, the recombinant or engineered viral vectors comprise from 5′ to 3′: a first inverted terminal repeat sequence, a first transcription termination sequence, an expression cassette, a second inverted terminal repeat sequence, and a second transcription termination sequence. In some embodiments, the recombinant or engineered viral vectors comprise from 5′ to 3′: a first transcription termination sequence, a first inverted terminal repeat sequence, an expression cassette, a second inverted terminal repeat sequence, and a second transcription termination sequence.

In select embodiments, the viral vector comprises from 5′ to 3′: a first or 5′ inverted terminal repeat sequence, a transcription termination sequence, an expression cassette, and a second or 3′ inverted terminal repeat sequence.

In some embodiments, the viral vector further comprises a polyadenylation (Poly(A)) signal sequence downstream of the expression cassette. For example, the viral vector comprises from 5′ to 3′: a first or 5′ inverted terminal repeat sequence adjacent either upstream (5′) or downstream (3′) to a first transcription termination sequence, an expression cassette, a Poly(A) signal sequence, and a second or 3′ inverted terminal repeat sequence adjacent either upstream (5′) or downstream (3′) to a second transcription termination sequence. In select embodiments, the viral vector comprises from 5′ to 3′: a first or 5′ inverted terminal repeat sequence, a first transcription termination sequence, an expression cassette, a Poly(A) signal sequence, and a second or 3′ inverted terminal repeat sequence.

In some embodiments, the viral vector is derived from a virus in the Parvoviridae family. Parvoviridae comprise a family of single-stranded DNA animal viruses. The parvoviruses and other members of the Parvoviridae family are generally described in Kenneth I. Berns, “Parvoviridae: The Viruses and Their Replication,” Chapter 69 in FIELDS VIROLOGY (3d Ed. 1996). Parvoviridae viruses include, for example, parvoviruses (e.g., chicken parvovirus, feline panleukopenia virus, hb parvovirus, h-1 parvovirus, killham rat virus, lapine parvovirus, luiii virus, minute virus of mice, mouse parvovirus 1, porcine parvovirus, rt parvovirus, tumor virus x, hamster parvovirus, rat minute virus 1, and rat parvovirus 1), erythroviruses (e.g., human parvovirus b19, pig-tailed macaque parvovirus, rhesus macaque parvovirus, simian parvovirus, bovine parvovirus type 3, and chipmunk parvovirus), dependoviruses (e.g., AAV-1, AAV-2, AAV-3, AAV-4, AAV-5, avian AAV, bovine AAV, canine AAV, duck AAV, equine AAV, goose parvovirus, ovine AAV, AAV-7, AAV-8, and bovine parvovirus 2), amdoparvoviruses (e.g., Aleutian mink disease virus), bocaviruses (e.g., bovine parvovirus and canine minute parvovirus), densoviruses (e.g., Galleria mellonella densovirus, Junonia coenia densovirus, Diatraea saccharalis densovirus, Pseudoplusia includens densovirus, and Toxorhynchites splendens densovirus), iteraviruses (e.g., Bombyx mori densovirus, Casphalia extranea densovirus, and Sibine fusca densovirus), brevidensoviruses (e.g., Aedes aegypti densovirus and Aedes albopictus densovirus), and pefudensoviruses (e.g., Periplaneta fuliginosa densovirus).

In some embodiments, the vector is derived from an adeno-associated virus (AAV). By adeno-associated virus, or “AAV” it is meant the virus itself or derivatives thereof. The term covers all subtypes and both naturally occurring and recombinant forms, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, a hybrid AAV (e.g., an AAV comprising a capsid protein of one AAV subtype and genomic material of another subtype), an AAV comprising a mutant AAV capsid protein or a chimeric AAV capsid (e.g., a capsid protein with regions or domains or individual amino acids that are derived from two or more different serotypes of AAV, e.g. AAV-DJ, AAV-LK3, AAV-LK19). “Primate AAV” refers to AAV that infect primates, “non-primate AAV” refers to AAV that infect non-primate mammals, “bovine AAV” refers to AAV that infect bovine mammals, etc.

Thus, in some embodiments, the source of vector components described herein, may be readily selected from among any AAV. These components may be readily isolated from an AAV sequence using techniques available to those of skill in the art of vector genome generation. Such parental AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

### A. Transcription Termination Sequence

The viral vectors described herein comprise a transcription termination sequence downstream of a first ITR preceding the expression cassette in order to prevent unwanted expression of the gene products of interest from the expression cassette. The transcription termination sequence may comprise any sequence that hinders or stops transcription by a polymerase.

Termination sequences can vary between different RNA polymerases and between eukaryotic and prokaryotic systems. In eukaryotes, RNA Polymerase III terminates transcription at T-rich sequences and seems to involve a limited number of auxiliary factors. RNA Polymerase I terminates at a major terminator (e.g., an 18-bp DNA sequence element referred to as the “Sal box”) and requires terminator recognition by specific protein factors (Lang and Reeder Mol Cell Biol. 1993 January; 13 (1): 649-658). For RNA Polymerase II, there are two broad categories of terminator sequence; G-rich sequences that enhance Pol II termination by pausing Pol II and AT-rich terminator sequences, which mediate rapid co-transcriptional cleavage of nascent transcripts (Richard, P., and Manley, J. L. Genes Dev. 2009; 23, 1247-1269).

Commonly used mammalian terminators (e.g., SV40, hGH, BGH, and rbGlob) include the sequence motif AATAAA which promotes both polyadenylation and termination. SV40 late polyA and rbGlob polyA are thought to be more efficient in terminating transcription due to the presence of additional helper sequences.

In some embodiments, the transcription termination sequence comprises one or more copies (e.g., one, two, three, four, five, six, seven, eight, nine, ten, or more copies) of a Poly(A) signal sequence comprising the characteristic AATAAA hexanucleotide motif. Any known Poly(A) signal sequence may be utilized between the first ITR and expression cassette. For example, the Poly(A) signal sequence may be selected from the polyadenylation signal sequences of: simian virus 40 (SV40), a-globin, b-globin, human collagen, human growth hormone (hGH), polyoma virus, human growth hormone (hGH) and bovine growth hormone (bGH).

In some embodiments, the Poly(A) signal sequence is a shortened Poly(A) signal sequence compared to the majority of Poly(A) sequences, which can range from 200-500 bases, on average. In some embodiments, the Poly(A) signal sequence may be or be derived from the sNRP-1 dual-functional stop codon/Poly(A) signal sequence (See, for example, McFarland, T J, et al., Plasmid 56 (2006) 62-67, incorporated herein by reference). Other short Poly(A) signal sequences have been identified and are known in the art, including, for example, the short polyA signal from the Bombyx moribidensovirus (Sec, Wang, M, et al., Invertebrate Survival Journal Vol. 14 No. 1 (2017), incorporated herein by reference).

### B. Inverted Terminal Repeat Sequences

Inverted terminal repeat or ITR sequences are sequences which can form a hairpin structure that mediate proviral integration and packaging of the vector into virions. ITR sequences also have roles in replication of the vector genome and integration and rescue from a host cell genome.

The ITR sequences may be isolated or derived from the genome of any virus. In some embodiments, one or both of the first and second ITR sequences are derived from a member of the Parvoviridae family. In select embodiments, one or both of the first and second ITR sequences are derived from an adeno-associated virus. The ITR sequences of any adeno-associated virus (AAV) may be suitable for use in the disclosed vectors, including but not limited to those sequence from the genomes of: AAV1, AAV2, AAV3, AAV4, AAV5, AAV6, AAV7, AAV8, AAV9, AAV10, AAV11, AAV12, AAVrh8, AAVrhIO, AAVrh32.33, AAVrh74, Avian AAV or Bovine AAV. In some embodiments, at least one of the first and second ITR may be derived from a member of another parvovirus species besides AAV. In some embodiments, one or both of the first and second ITR sequences may be wild-type sequences of the virus from which it was derived.

In some embodiments, one or both of the first and second ITR sequences may be engineered. In some embodiments, the engineered ITR sequence may comprise one or more nucleotide substitutions, additions, or deletions as compared to the wild-type sequence. In some embodiments, the one or more nucleotide substitutions, additions, or deletions promote production of a virus or viral particle. In some embodiments, the one or more nucleotide substitutions, additions, or deletions comprise a deletion of the terminal resolution sequence (TRS) from the ITR. In some embodiments, the terminal resolution sequence is absent from both the 5′ ITR and the 3′ITR. In some embodiments, the engineered ITR sequence may comprise a sequence that has little or no sequence relationship to a wild-type ITR but serves the same function as an ITR. Sec, for example, Xie, J. et al., Mol. Then, 25 (6): 1363-1374 (2017).

The ITR sequences may be about 100 to about 200 nucleotides in length, for example about 100, about 110, about 120, about 130, about 140, about 150, about 160, about 170, about 180, about 190, or about 200 nucleotides in length. The first ITR sequence and the second ITR sequence may be the same or different in length. For example, the first ITR sequence may be longer than the second ITR sequence or, conversely, the second ITR sequence may be longer than the first ITR sequence.

c. Expression Cassette

The expression cassette essentially comprises the sequences which, when the vector is introduced into a cell, direct the machinery of the cell to make any or all of the gene products of interest encoded in the expression cassette.

In some embodiments, the vector comprises an expression cassette having one or more sequences which facilitate introduction of regulatory control elements and sequences encoding one or more gene products (e.g., protein, RNA) of interest into the vector. Thus, the expression cassette is configured to receive, for example, by standard molecular cloning techniques, the desired regulatory control elements and sequences encoding the gene products of interest. For example, the expression cassette may comprise one or more versatile cloning sites (e.g., restriction endonuclease recognition sequences) by which the desired regulatory sequences and sequences encoding the gene products of interest can be introduced.

Alternatively, in some embodiments, the vector comprises an expression cassette having one or more regulatory control elements and one or more sequences (e.g., versatile cloning sites (e.g., restriction endonuclease recognition sequences)) which facilitate introduction of the desired sequences encoding the gene products of interest.

Alternatively, in some embodiments, the vector comprises an expression cassette having a heterologous nucleic acid sequence encoding one or more gene products (e.g., protein, RNA) of interest operably linked to one or more regulatory control elements. The term “gene product,” as used herein, refers to any biochemical product resulting from expression of a gene. Gene products may be RNA or protein. In some embodiments, the expression cassette encodes an RNA. RNA gene products include, but are not limited to, non-coding RNA, such as tRNA, rRNA, micro RNA (miRNA), and small interfering RNA (siRNA), and coding RNA, such as messenger RNA (mRNA). In some embodiments, the expression cassette encodes a protein, polypeptide, or peptide. In some embodiments, the one or more gene products of interest include one or more proteins, polypeptides, or peptides, or one or more RNAs, or a combination thereof.

When used in mammalian cells, the control functions of the vector are typically provided by the one or more regulatory control elements. For example, vectors of the present disclosure can comprise any of a number of promoters known to the art, wherein the promoter is constitutive, regulatable or inducible, cell type specific, tissue-specific, or species specific. In addition to the promoter, a regulatory control element of the invention can also include other regulatory elements that are involved in modulating transcription (e.g., enhancers, Kozak sequences and introns).

Many promoter/regulatory control elements useful for driving constitutive expression of a gene are available in the art and include, but are not limited to, for example, CMV (cytomegalovirus) promoter, EF1a (human elongation factor 1 alpha) promoter, SV40 (simian vacuolating virus 40) promoter, PGK (mammalian phosphoglycerate kinase) promoter, Ubc (human ubiquitin C) promoter, human beta-actin promoter, rodent beta-actin promoter, CBh (chicken beta-actin) promoter, CAG promoter (hybrid promoter containing CMV enhancer, chicken beta actin promoter, and rabbit beta-globin splice acceptor), TRE (Tetracycline response element) promoter, H1 (human polymerase III RNA) promoter, U6 (human U6 small nuclear) promoter, and the like. Additional promoters that can be used include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, mycoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Inducible and tissue specific expression of an RNA and/or protein can be accomplished by placing the nucleic acid encoding such a molecule under the control of an inducible or tissue specific promoter/regulatory control sequences. Examples of tissue specific or inducible promoter/regulatory control sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoter/regulatory control sequences and tumor-specific are available, for example from InvivoGen. In addition, promoter/regulatory control sequences which are known in the art can be induced in response to inducing agents such as metals, glucocorticoids, tetracycline, hormones, and the like, are also contemplated for use with the invention. Thus, it will be appreciated that the present disclosure includes the use of any promoter/regulatory control sequences known in the art that is capable of driving expression of the desired gene product operably linked thereto.

The vectors of the present disclosure may direct expression in a particular cell type (e.g., using tissue-specific regulatory elements). Such regulatory control elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

d. Polyadenylation Sequence

As described above, in some embodiments, the viral vector further comprises a polyadenylation (Poly(A)) signal sequence downstream of the expression cassette. The polyadenylation signal sequence controls the addition of a string of adenosine residues (the Poly(A) tail) to the 3′ end of the gene transcript and generally includes an AATAAA hexanucleotide motif. Any known Poly(A) signal sequence may be utilized downstream of the expression cassette. For example, the Poly(A) signal sequence may be selected from the polyadenylation signal of simian virus 40 (SV40), a-globin, b-globin, human collagen, human growth hormone (hGH), polyoma virus, human growth hormone (hGH) and bovine growth hormone (bGH). The Poly(A) signal sequence may be any length. Common polyadenylation signal sequences, such as the SV40 pA and the human growth hormone (hGH) pA, can range in length from 250 to 450 bases.

Additionally, the vector may contain, for example, some or all of the following: a selectable marker gene, such as the neomycin gene for selection of stable or transient transfectants in host cells; 5′- and 3′-untranslated regions for mRNA stability and translation efficiency from highly-expressed genes like a-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression.

In some instances, the recombinant viral vector also comprises viral genes important for the packaging of the recombinant viral vector material. Packaging refers to the series of intracellular events that result in the assembly and encapsulation of a viral particle, e.g., an AAV viral particle. Examples of nucleic acid sequences important for AAV packaging include the AAV “rep” and “cap” genes, which encode for replication and encapsulation proteins of adeno-associated virus, respectively.

Viral vectors typically have a defined size range in which the vector is viable. For example, AAV vectors typically accept DNA inserts from about 4 kb to about 5.2 kb, or slightly more. In instances when the sequence encoding the gene product(s) of interest are shorter, additional nucleic acids may be needed to achieve the length acceptable for the viral vector. As such, the viral vector may comprise a filler sequence of variable length to make up the difference. The filler sequence can be located at any position that does not prevent or retard the function, activity, or packaging of the vector.

Also provided herein are methods for decreasing unwanted gene expression from a viral vector comprising introducing a transcription termination sequence downstream of an ITR sequence and upstream of an expression cassette. The transcription termination sequence useful in these methods are any of those described above. Descriptions of ITR sequences and expression cassettes provided above are equally applicable to such methods.

### Virus or Virus-Like Particles

Provided herein are virus and virus-like particles (VLPs) comprising the viral vectors disclosed herein. A “virus particle” refers to a single unit of virus comprising a capsid encapsulating a polynucleotide, e.g., the viral genome (as in a wild-type virus) or a vector, such as those disclosed herein, (as in a recombinant virus). “Virus-like particles” or “VLPs” refer to a structure that in at least one attribute resembles a virus but which has not been demonstrated to be infectious. Virus-like particles may or may not carry genetic information encoding for the proteins of the virus-like particle, but in general do not include the genetic materials required for viral replication and infection.

In some embodiments, the virus or virus-like particle is derived from a parvovirus, as described above. In some embodiments, the virus or virus-like particle is derived from an AAV virus. An AAV virus particle refers to a viral particle composed of at least one AAV capsid protein (typically by all of the capsid proteins of a wild-type AAV). Given the particle comprises a heterologous viral vector, as described above, it would be referred to as an “rAAV vector particle.”

A rAAV virion can be constructed a variety of methods. For example, the heterologous sequence(s) can be directly inserted into an AAV genome which has had the major AAV open reading frames (“ORFs”) excised therefrom. Other portions of the AAV genome can also be deleted, so long as a sufficient portion of the ITRs remain to allow for replication and packaging functions. In order to produce rAAV virions, an AAV expression vector can be introduced into a suitable host cell using known techniques, such as by transfection. Particularly suitable transfection methods include calcium phosphate co-, direct micro-injection into cultured cells, electroporation, liposome mediated gene transfer, lipid-mediated transduction, and nucleic acid delivery using high-velocity microprojectiles. Suitable cells for producing rAAV virions include microorganisms, yeast cells, insect cells, and mammalian cells, that can be, or have been, used as recipients of the viral vector.

An AAV virus that is produced may be replication competent or replication-incompetent. A “replication-competent” virus (e.g., a replication-competent AAV) refers to a phenotypically wild-type virus that is infectious and is also capable of being replicated in an infected cell (e.g., in the presence of a helper virus or helper virus functions). In the case of AAV, replication competence generally requires the presence of functional AAV packaging genes. In general, rAAV vectors as described herein are replication-incompetent in mammalian cells, particularly in human cells, by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector.

### Compositions

Further disclosed herein are compositions comprising the disclosed vectors, viruses, viral particles, and virus-like particles. In some embodiments, the composition comprises a carrier, e.g., a pharmaceutically acceptable carrier. The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopcia for use in mammals, and more particularly in humans. “Acceptable” means that the carrier is compatible with the viral vector or virus or virus-like particle and does not negatively affect the subject to which the composition(s) are administered.

Carriers may include any and all solvents, dispersion media, coatings, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, coating agents, sweetening, flavoring and perfuming agents, preservatives and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

### Methods

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector. Additionally, delivery vehicles such as nanoparticle- and lipid-based delivery systems can also be used to deliver the viral vector to cells.

As such, also provided herein are methods comprising delivering the disclosed viral vectors to a cell. In some embodiments, the methods comprise contacting the cells with an effective amount of a recombinant viral vector, a virus or virus-like particle, or a pharmaceutical composition, as described herein. As used herein, in some embodiments, an effective amount is an amount of the viral vector that introduces sufficient amounts of the nucleic acid of the expression cassette to confer the intended function, e.g., to result in production of the desired gene product(s) in the cell of interest. In general, the viral vectors described herein can be employed to deliver any heterologous nucleic acid, and accordingly any desired gene product, to a cell of interest.

The viral vectors, virus or virus-like particles, and pharmaceutical compositions disclosed herein provide a means for delivering nucleic acids and thus gene products into a broad range of cells, including dividing and non-dividing cells. The viral vectors, virus or virus-like particles, and pharmaceutical compositions can be employed to deliver to a cell in vitro, e.g., to produce a polypeptide in vitro or for ex vivo gene therapy.

The cell may be, a plant cell, an insect cell, a vertebrate cell, an invertebrate cell, an animal cell, a mammalian cell, or a human cell. In some embodiments, the cell is a plant cell. In some embodiments, the cell is an insect cell. In some embodiments, the cell is a vertebrate cell. In some embodiments, the cell is an invertebrate cell. In some embodiments, the cell is a mammalian cell. In some embodiments, the cell is a human cell. In some embodiments, the cell is a stem cell. In some cases, the cell is ex vivo (e.g., fresh isolate-early passage). In some cases, the cell is in vivo. In some cases, the cell is in culture in vitro (e.g., immortalized cell line).

The viral vectors, virus or virus-like particles, and pharmaceutical compositions are additionally useful in methods of delivering a gene product to cells in a subject, e.g., to express an immunogenic or therapeutic polypeptide or a functional RNA in the subject. The subject can be in need because the subject has a deficiency of the gene product or the production of the gene product in the subject may impart some beneficial effect, e.g., therapeutic or prophylactic benefit. More specifically, the viral vectors, virus or virus-like particles, and pharmaceutical compositions described herein can be used to deliver a desired gene product (e.g., polypeptide, protein, or functional RNA) to treat and/or prevent a disease state for which it is therapeutically or prophylactically beneficial to administer the gene product. For example, the expression cassette may encode therapeutic (e.g., for medical, veterinary uses) or immunogenic (e.g., for vaccines) polypeptides proteins, or RNAs.

In some embodiments, the expression cassette may encode one or more RNAs, including for example, an antisense nucleic acid, a ribozyme, RNAs that effect spliceosome-mediated/ram-splicing, interfering RNAs (RNAi) including siRNA, shRNA or miRNA that mediate gene silencing, and other non-translated RNAs.

Alternatively, or in addition, in some embodiments, the expression cassette may encode one or more protein or polypeptides. Useful therapeutic protein or polypeptide products encoded by the expression cassette include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15 as well as TGFb proteins, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful gene products include proteins that regulate the immune system including, without limitation, cytokines and lymphokines such as thrombopoietin (TPO), interleukins (IL) IL-1 through IL-25 (e.g., IL-2, IL-4, IL-12, and IL-18), monocyte chemoattractant protein, leukemia inhibitory factor, granulocyte-macrophage colony stimulating factor, Fas ligand, tumor necrosis factors α and β, interferons α, β, TGFb and γ, stem cell factor, flk-2/flt3 ligand. Gene products produced by the immune system, or recombinant and engineered forms thereof, are also useful in the disclosed methods. These include, without limitation, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. For example, receptors for cholesterol regulation and/or lipid modulation, including the low density lipoprotein (LDL) receptor, high density lipoprotein (HDL) receptor, the very low density lipoprotein (VLDL) receptor, and scavenger receptors; glucocorticoid receptors and estrogen receptors; Vitamin D receptors; and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

In some embodiments, the viral vectors, virus or virus-like particles, and pharmaceutical compositions described herein can be used to deliver a gene editing system. Thus, in some embodiments, the expression cassette encodes a gene editing system or a component thereof. The gene editing system may be used to modulate target gene expression (e.g., activate or repress a target gene), introduce one or more nucleotide substitutions, addition, or deletions into a target gene (e.g., to insert or correct a mutation, insert a tag, disrupt target gene expression, or alter a promoter region for a target gene), or delete a target gene. For example, the expression cassette may encode a zinc-finger nuclease, a homing endonuclease, a TALEN (transcription activator-like effector nuclease), a NgAgo (agronaute endonuclease), a SGN (structure-guided endonuclease), or one or more components of a CRISPR-Cas system.

A “CRISPR-Cas system” refers collectively to transcripts and other elements involved in the expression of and/or directing the activity of CRISPR-associated (“Cas”) genes, including sequences encoding a Cas gene, Cas protein, a cr (CRISPR) sequence (e.g., crRNA or an active partial crRNA), or other sequences and transcripts from a CRISPR locus. In some embodiments, one or more elements of a CRISPR system is derived from a type I, type II, or type III CRISPR system. The CRISPR-Cas system can be an engineered system for use in activation, repression, or deletion of a target gene (e.g., CRISPRi, CRISPRa). CRISPR-Cas editing technology is described in detail in, for example, U.S. Pat. Nos. 8,546,553, 8,697,359; 8,771,945; 8,795,965; 8,865,406; 8,871,445; 8,889,356; 8,889,418; 8,895,308; 8,9066,616; 8,932,814; 8,945,839; 8,993,233; 8,999,641; 9,115,348; 9,149,049; 9,493,844; 9,567,603; 9,637,739; 9,663,782; 9,404,098; 9,885,026; 9,951,342; 10,087,431; 10,227,610; 10,266,850; 10,601,748; 10,604,771; and 10,760,064; and U.S. Patent Application Publication Nos. US2010/0076057; US2014/0113376; US2015/0050699; US2015/0031134; US2014/0357530; US2014/0349400; US2014/0315985; US2014/0310830; US2014/0310828; US2014/0309487; US2014/0294773; US2014/0287938; US2014/0273230; US2014/0242699; US2014/0242664; US2014/0212869; US2014/0201857; US2014/0199767; US2014/0189896; US2014/0186919; US2014/0186843; and US2014/0179770, each incorporated herein by reference.

For example, the gene editing system may comprise one or more Cas proteins (e.g., Cas9), or other RNA-guided nucleases, and at least one guide RNA directed to a target nucleic acid. Typically, the RNA sequences employed in CRISPR/Cas systems are referred to collectively as “guide RNA” (gRNA) or single guide RNA (sgRNA). Thus, the terms “guide RNA,” “single guide RNA,” and “synthetic guide RNA,” are used interchangeably herein and may refer to a nucleic acid sequence comprising a tracrRNA and a pre-crRNA array containing a guide sequence. The terms “guide sequence,” “guide,” and “spacer,” are used interchangeably herein and refer to the nucleotide sequence within a guide RNA that specifies the target nucleic acid.

Alternatively, or in addition to any of the above, the expression cassette may comprise a sequence which shares homology and recombines with a nucleic acid in the host cell, for example, on a host chromosome. This approach find use, for example, in correcting a genetic defect in the host cell.

### Systems or Kits

Also within the scope of the present disclosure are systems or kits comprising a viral vector as described herein. The systems or kits may further comprise one or more of: buffer or carrier constituents, restriction endonucleases or related materials for molecular cloning, control vectors, sequencing primers, transfection reagents, and cells for making the virus or virus-like particles, or expression of the gene products.

The kit may include instructions for use in any of the methods described herein. The instructions can comprise a description of administration of the viral vectors, virus or virus-like particles, or compositions to a subject to achieve the intended effect. The instructions generally include information as to dosage and administration.

The kits provided herein are in suitable packaging. Suitable packaging includes, but is not limited to, vials, bottles, jars, flexible packaging, and the like. Normally, the kit comprises a label or package insert(s) on or associated with the packaging. The packaging may be unit doses, bulk packages (e.g., multi-dose packages) or sub-unit doses. Instructions supplied in the kits of the disclosure are typically written instructions on a label or package insert.

### EXAMPLES

The following are examples of the present invention and are not to be construed as limiting.

### Example 1

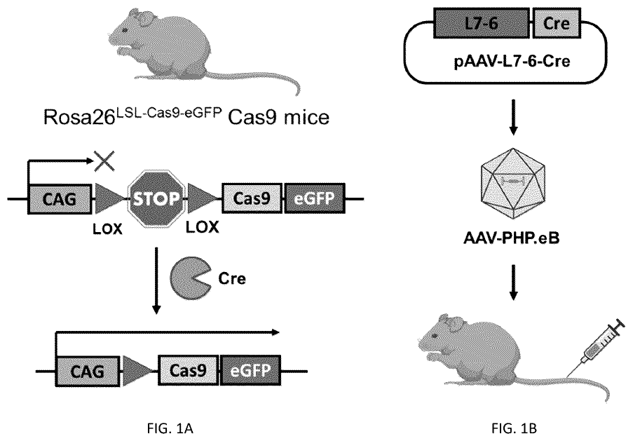

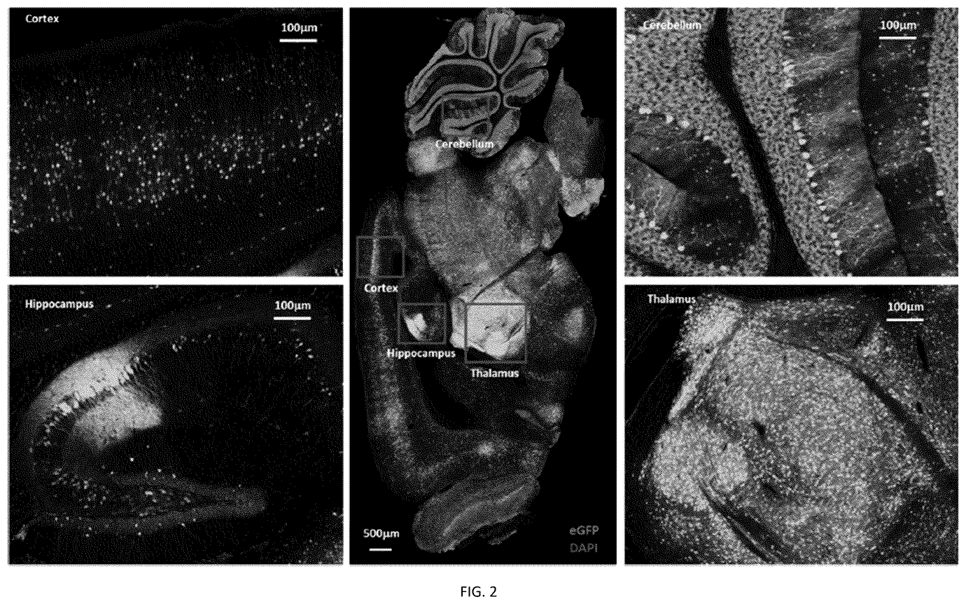

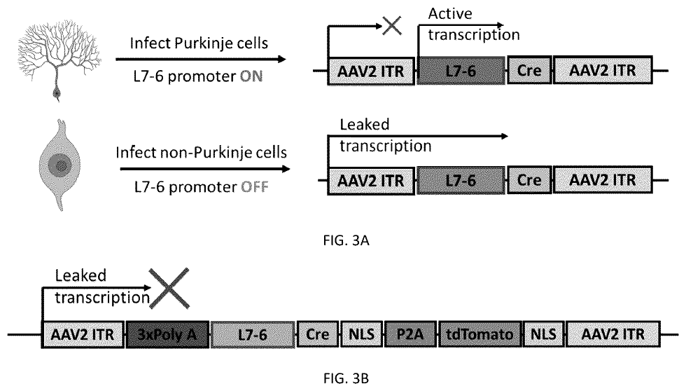

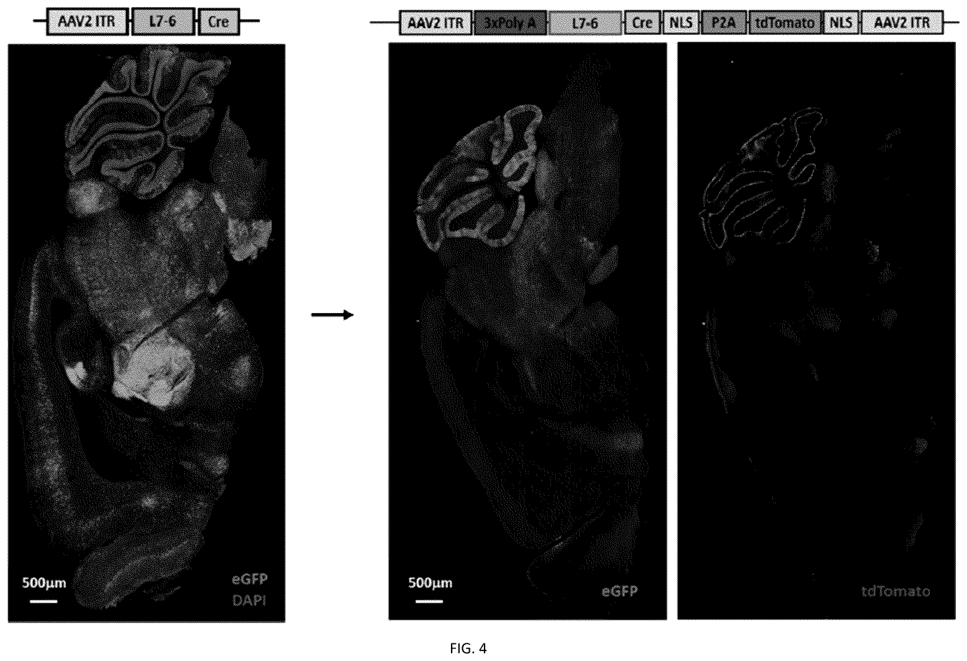

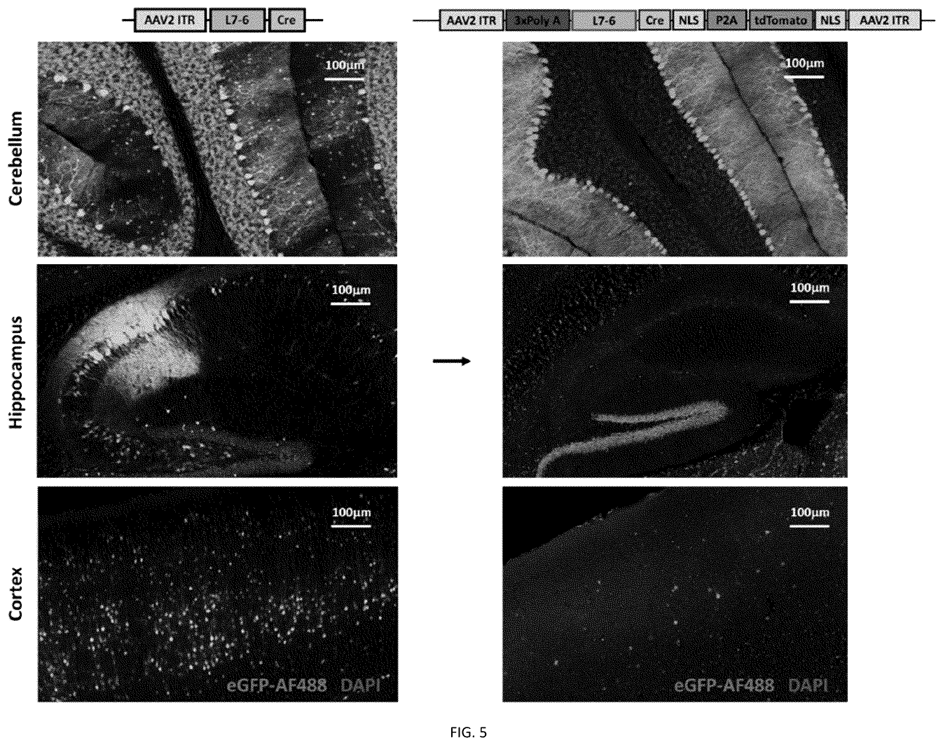

To achieve Purkinje cell specific expression of the Cas9 nuclease, a Rosa26LSL-Cas9 “Cas9” mouse model (available from Jackson laboratories) which contains the Cas9 gene downstream of a Lox-Stop-Lox (LSL) transcriptional termination cassette was utilized. In this mouse model, Cas9 will only be expressed upon CRE-mediated excision of the LSL sequence (FIG. 1A). The CRE recombinase is easier to deliver in vivo than Cas9 given that it is much smaller enabling it to be efficiently packaged along with the required regulatory sequences into adeno-associated virus (AAV) made with the PHP.eB serotype, which is able to readily infect Purkinje cells when injected intravenously (FIG. 1B). To focus the studies on Purkinje cells the expression of CRE is driven by a Purkinje cell specific promoter, L7-6, which then restrict Cas9 expression and gene knockout events to only Purkinje cells (FIG. 1B). In testing the designed system, a large amount of non-specific CRE activity was observed in numerous cells throughout the brain including the cortex, hippocampus, including various non-Purkinje cells within the cerebellum (the area of the brain in which Purkinje cells are located) (FIG. 2). These results demonstrated that LoxP excision mediated by CRE is very sensitive and thus any amount of off target CRE expression in non-Purkinje cells will be enough to remove the LSL element and lead to Cas9 expression in those cells. Given the previous literature suggesting that the L7-6 promoter was specific, it was hypothesized that the inappropriate gene expression being observed may have been arising from the ITR elements present at the ends of the AAV vector and which were upstream of the L7-6 promoter (FIG. 3A). Therefore, to eliminate the effect of ITR mediated transcription of CRE in non-Purkinje cells, a 3×Poly(A) transcriptional termination cassette was inserted between the ITR and L7-6 promoter (FIG. 3B). As designed this method greatly reduced the inappropriate CRE expression from the vast majority of non-Purkinje cells. (FIGS. 4 and 5).

pAAV-sgRNA-Lox-watermark-Lox-UMI (SEQ ID NO: 1):
cctgcaggcagctgcgcgctcgctcgctcactgaggccgcccgggcgtcgggcgacctttggtcgcccggcctcagtgagcgagcgag
cgcgcagagagggagtggccaactccatcactaggggttcctgcggccgcccccttcaccgagggcctatttcccatgattccttcatatttg
catatacgatacaaggctgttagagagataattggaattaatttgactgtaaacacaaagatattagtacaaaatacgtgacgtagaaagtaat
aatttcttgggtagtttgcagttttaaaattatgttttaaaatggactatcatatgcttaccgtaacttgaaagtatttcgatttcttggctttatata
tcttgtggaaaggacgaaacaccggagacgattaatgcgtctcggttttagagctagaaatagcaagttaaaataaggctagtccgttatcaacttg
aaaaagtggcaccgagtcggtgctttttttATAACTTCGTATAGCATACATTATACGAAGTTATggcgcgcc
actggatccgctcgtacgtagaagcttccgccgcagtctcacgcccggagcgtagcgaccgagtgagctagctatttgtttatttttctaaata
cattcaaatatgtatccgctcatgagacaataaccctgataaatgcttcaataatattgaaaaaggaagagtatgagggaagcggtgatcgcc
gaagtatcgactcaactatcagaggtagttggcgtcatcgagcgccatctcgaaccgacgttgctggccgtacatttgtacggctccgcagt
ggatggcggcctgaagccacacagtgatattgatttgctggttacggtgaccgtaaggcttgatgaaacaacgcggcgagctttgatcaacg
accttttggaaacttcggcttcccctggagagagcgagattctccgcgctgtagaagtcaccattgttgtgcacgacgacatcattccttggcg
ttatccagctaagcgcgaactgcaatttggagaatggcagcgcaatgacattcttgcgggtatcttcgagccagccacgatcgacattgatct
ggctatcttgctgacaaaagcaagagaacatagcgttgccttggtaggtccagcggcggaggaactctttgatccggttcctgaacaggatc
tatttgaggcgctaaatgaaaccttaacgctatggaactcgccgcccgactgggctggcgatgagcgaaatgtagtgcttacgttgtcccgc
atttggtacagcgcagtaaccggcaaaatcgcgccgaaggatgtcgctgccgactgggcaatggagcgcctgccggcccagtatcagcc
cgtcatacttgaagctagacaggcttatcttggacaagaagaagatcgcttggcctcgcgcgcagatcagttggaagaatttgtccactacgt
gaaaggcgagatcaccaaggtagtcggcaaataaATAACTTCGTATAGCATACATTATACGAAGTTATN
NNNNNNNgaataaaacgcacgggtgttgaattcgcggccgcaggaacccctagtgatggagttggccactccctctctgcgcgctc
gctcgctcactgaggccgggcgaccaaaggtcgcccgacgcccgggctttgcccgggcggcctcagtgagcgagcgagcgcgcagct
gcctgcaggggcgcctgatgcggtattttctccttacgcatctgtgcggtatttcacaccgcatacgtcaaagcaaccatagtacgcgccctg
tagcggcgcattaagcgcggcgggtgtggtggttacgcgcagcgtgaccgctacacttgccagcgccctagcgcccgctcctttcgctttct
tcccttcctttctcgccacgttcgccggctttccccgtcaagctctaaatcgggggctccctttagggttccgatttagtgctttacggcacctcg
accccaaaaaacttgatttgggtgatggttcacgtagtgggccatcgccctgatagacggtttttcgccctttgacgttggagtccacgttcttta
atagtggactcttgttccaaactggaacaacactcaaccctatctcgggctattcttttgatttataagggattttgccgatttcggcctattggtta
aaaaatgagctgatttaacaaaaatttaacgcgaattttaacaaaatattaacgtttacaattttatggtgcactctcagtacaatctgctctgatgc
cgcatagttaagccagccccgacacccgccaacacccgctgacgcgccctgacgggcttgtctgctcccggcatccgcttacagacaagc
tgtgaccgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattgaaa
aaggaagagtatgagtattcaacatttccgtgtcgcccttattcccttttttgcggcattttgccttcctgtttttgctcacccagaaacgctggtga
aagtaaaagatgctgaagatcagttgggtgcacgagtgggttacatcgaactggatctcaacagcggtaagatccttgagagttttcgcccc
gaagaacgttttccaatgatgagcacttttaaagttctgctatgtggcgcggtattatcccgtattgacgccgggcaagagcaactcggtcgcc
gcatacactattctcagaatgacttggttgagtactcaccagtcacagaaaagcatcttacggatggcatgacagtaagagaattatgcagtg
ctgccataaccatgagtgataacactgcggccaacttacttctgacaacgatcggaggaccgaaggagctaaccgcttttttgcacaacatg
ggggatcatgtaactcgccttgatcgttgggaaccggagctgaatgaagccataccaaacgacgagcgtgacaccacgatgcctgtagca
atggcaacaacgttgcgcaaactattaactggcgaactacttactctagcttcccggcaacaattaatagactggatggaggcggataaagtt
gcaggaccacttctgcgctcggcccttccggctggctggtttattgctgataaatctggagccggtgagcgtgggtctcgcggtatcattgca
gcactggggccagatggtaagccctcccgtatcgtagttatctacacgacggggagtcaggcaactatggatgaacgaaatagacagatc
gctgagataggtgcctcactgattaagcattggtaactgtcagaccaagtttactcatatatactttagattgatttaaaacttcatttttaatttaa
aaggatctaggtgaagatcctttttgataatctcatgaccaaaatcccttaacgtgagttttcgttccactgagcgtcagaccccgtagaaaagat
caaaggatcttcttgagatcctttttttctgcgcgtaatctgctgcttgcaaacaaaaaaaccaccgctaccagcggtggtttgtttgccggatca
agagctaccaactctttttccgaaggtaactggcttcagcagagcgcagataccaaatactgtccttctagtgtagccgtagttaggccacca
cttcaagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacggggggttcgtgcacacagcccagcttggagcgaacgac
ctacaccgaactgagatacctacagcgtgagctatgagaaagcgccacgcttcccgaagggagaaaggcggacaggtatccggtaagc
ggcagggtcggaacaggagagcgcacgagggagcttccagggggaaacgcctggtatctttatagtcctgtcgggtttcgccacctctga
cttgagcgtcgatttttgtgatgctcgtcaggggggcggagcctatggaaaaacgccagcaacgcggcctttttacggttcctggccttttgct
ggccttttgctcacatgt
pAAV-3xPolyA-L7-6-Cre-P2A-tdTomato-NLS (SEQ ID NO: 2):
cctgcaggcagctgcgcgctcgctcgctcactgaggccgcccgggcgtcgggcgacctttggtcgcccggcctcagtgagcgagcgag
cgcgcagagagggagtggccaactccatcactaggggttcctgcggccgcacgcgttgcagatctgcgactctagaggatctgcgactct
agaggatcataatcagccataccacattttgtagaggttttacttgctttaaaaaacctcccacacctccccctgaacctgaaacataaaatgaa
tgcaattgttgttgttaacttgtttattgcagcttataatggttacaaataaagcaatagcatcacaaatttcacaaataaagcatttttttcactgc
attctagttgtggtttgtccaaactcatcaatgtatcttatcatgtctggatctgcgactctagaggatcataatcagccataccacatttgtagaggt
tttacttgctttaaaaaacctcccacacctccccctgaacctgaaacataaaatgaatgcaattgttgttgttaacttgtttattgcagcttataatgg
ttacaaataaagcaatagcatcacaaatttcacaaataaagcatttttttcactgcattctagttgtggtttgtccaaactcatcaatgtatcttatca
tgtctggatctgcgactctagaggatcataatcagccataccacatttgtagaggttttacttgctttaaaaaacctcccacacctccccctgaac
ctgaaacataaaatgaatgcaattgttgttgttaacttgtttattgcagcttataatggttacaaataaagcaatagcatcacaaatttcacaaataa
agcatttttttcactgcattctagttgtggtttgtccaaactcatcaatgtatcttatcatgtctggatccTTAATtaagtgtctagaggttccac
cctcatgttggttgatcttcaacattgtcctgacttcttcccacttgacattcctcagggtcctgtgatcatggctgggtctagtgaggttcaaacc
tgcactgccctacccacacccacacccagctcagcgtcagtcaggatcaacagttacctagagatcatctttctggggcttaagcattggtgg
gagcagatgggatatgagttggggatttggggatgggggaagatatctgctccccctccccctacaccctagccttttaaaaggccttctcag
gtcagagaccaggagaaaagtataggagagatacacaatggaccaggaagaagaaaagggagagggaggctcagaccttctagacaa
ggtaagagggctctggctgactccaccatccgcttcttgaggtctcggcacctgtaattgacaagattaattcatttatagggcatctaattagc
aagtaagtctctggagtcccctgacccagttactataacacacagggggtataggtaggggagtataagagcccctcctcagggcaaatga
atggattcttagtactgtcccccaagagatagtaggtactaggatttaggggcacttctgagccccatttccctggtaagtgtcccaacccccc
aaatcaacccaagcctggtctcaatctaggacagtggtagaatgctgtccctagagtcagtaccatgtgaaattgtgctgcaggcaggggcc
ccaggctgggaggtgggggttgggggagtcaggggcaggtcagggaaggagactcaggtttcatttagagaaaTTCTGCAGAC
CCGTGAGGACTggatccgccaccatggtgcccaagaagaagaggaaagtctccaacctgctgactgtgcaccaaaacctgcct
gccctccctgtggatgccacctctgatgaagtcaggaagaacctgatggacatgttcagggacaggcaggccttctctgaacacacctgga
agatgctcctgtctgtgtgcagatcctgggctgcctggtgcaagctgaacaacaggaaatggttccctgctgaacctgaggatgtgaggga
ctacctcctgtacctgcaagccagaggcctggctgtgaagaccatccaacagcacctgggccagctcaacatgctgcacaggagatctgg
cctgcctcgcccttctgactccaatgtgtgtccctggtgatgaggagaatcagaaaggagaatgtggatgctggggagagagccaagca
ggccctggcctttgaacgcactgactttgaccaagtcagatccctgatggagaactctgacagatgccaggacatcaggaacctggccttcc
tgggcattgcctacaacaccctgctgcgcattgccgaaattgccagaatcagagtgaaggacatctcccgcaccgatggtgggagaatgct
gatccacattggcaggaccaagaccctggtgtccacagctggtgtggagaaggccctgtccctgggggttaccaagctggtggagagatg
gatctctgtgtctggtgtggctgatgaccccaacaactacctgttctgccgggtcagaaagaatggtgtggctgccccttctgccacctccca
actgtccacccgggccctggaagggatctttgaggccacccaccgcctgatctatggtgccaaggatgactctgggcagagatacctggc
ctggtctggccactctgccagagtgggtgctgccagggacatggccagggctggtgtgtccatccctgaaatcatgcaggctggtggctgg
accaatgtgaacatagtgatgaactacatcagaaacctggactctgagactggggccatggtgaggctgctcgaggatggggacggaagc
ggagccactaacttctccctgttgaaacaagcaggggatgtcgaagagaatcccgggccaatggtgagcaagggcgaggaggtcatcaa
agagttcatgcgcttcaaggtgcgcatggagggctccatgaacggccacgagttcgagatcgagggcgagggcgagggccgcccctac
gagggcacccagaccgccaagctgaaggtgaccaagggcggccccctgcccttcgcctgggacatcctgtccccccagttcatgtacgg
ctccaaggcgtacgtgaagcaccccgccgacatccccgattacaagaagctgtccttccccgagggcttcaagtgggagcgcgtgatgaa
cttcgaggacggcggtctggtgaccgtgacccaggactcctccctgcaggacggcacgctgatctacaaggtgaagatgcgcggcacca
acttcccccccgacggccccgtaatgcagaagaagaccatgggctgggaggcctccaccgagcgcctgtacccccgcgacggcgtgct
gaagggcgagatccaccaggccctgaagctgaaggacggcggccactacctggtggagttcaagaccatctacatggccaagaagccc
gtgcaactgcccggctactactacgtggacaccaagctggacatcacctcccacaacgaggactacaccatcgtggaacagtacgagcgc
tccgagggccgccaccacctgttcctgtacggcatggacgagctgtacaagtccggaggaggacccaagaagaagaggaaggtgtagt
aaaagcttatcgataatcaacctctggattacaaaatttgtgaaagattgactggtattcttaactatgttgctccttttacgctatgtggatacgct
gctttaatgcctttgtatcatgctattgcttcccgtatggctttcattttctcctccttgtataaatcctggttgctgtctctttatgaggagttgtg
gcccgttgtcaggcaacgtggcgtggtgtgcactgtgtttgctgacgcaacccccactggttggggcattgccaccacctgtcagctcctttccgg
gactttcgctttccccctccctattgccacggcggaactcatcgccgcctgccttgcccgctgctggacaggggctcggctgttgggcactg
acaattccgtggtgttgtcggggaaatcatcgtcctttccttggctgctcgcctatgttgccacctggattctgcgcgggacgtccttctgctac
gtcccttcggccctcaatccagcggaccttccttcccgcggcctgctgccggctctgcggcctcttccgcgtcttcgccttcgccctcagacg
agtcggatctccctttgggccgcctccccgcatcgataccgagcgctgctcgagagatcttcgactgtgccttctagttgccagccatctgttg
tttgcccctcccccgtgccttccttgaccctggaaggtgccactcccactgtcctttcctaataaaatgaggaaattgcatcgcattgtctgagta
ggtgtcattctattctggggggtggggggggcaggacagcaagggggaggattgggaagacaatagcaggcatgagatctcacgtgcg
gaccgagcggccgcaggaacccctagtgatggagttggccactccctctctgcgcgctcgctcgctcactgaggccgggcgaccaaagg
tcgcccgacgcccgggctttgcccgggcggcctcagtgagcgagcgagcgcgcagctgcctgcaggggcgcctgatgcggtattttctc
cttacgcatctgtgcggtatttcacaccgcatacgtcaaagcaaccatagtacgcgccctgtagcggcgcattaagcgcggcgggtgtggt
ggttacgcgcagcgtgaccgctacacttgccagcgccttagcgcccgctcctttcgctttcttcccttcctttctcgccacgttcgccggctttc
cccgtcaagctctaaatcgggggctccctttagggttccgatttagtgctttacggcacctcgaccccaaaaaacttgatttgggtgatggttca
cgtagtgggccatcgccctgatagacggtttttcgccctttgacgttggagtccacgttctttaatagtggactcttgttccaaactggaacaac
actcaactctatctcgggctattcttttgatttataagggattttgccgatttcggtctattggttaaaaaatgagctgatttaacaaaaatttaacg
cgaattttaacaaaatattaacgtttacaattttatggtgcactctcagtacaatctgctctgatgccgcatagttacgccagccccgacacccgcc
aacacccgctgacgcgccctgacgggcttgtctgctcccggcatccgcttacagacaagctgtgaccgtctccgggagctgcatgtgtcag
aggttttcaccgtcatcaccgaaacgcgcgagacgaaagggcctcgtgatacgcctatttttataggttaatgtcatgataataatggtttctta
gacgtcaggtggcacttttcggggaaatgtgcgcggaacccctatttgtttatttttctaaatacattcaaatatgtatccgctcatgagacaata
accctgataaatgcttcaataatattgaaaaaggaagagtatgagtattcaacatttccgtgtcgcccttattcccttttttgcggcattttgccttc
ctgtttttgctcacccagaaacgctggtgaaagtaaaagatgctgaagatcagttgggtgcacgagtgggttacatcgaactggatctcaaca
gcggtaagatccttgagagttttcgccccgaagaacgttttccaatgatgagcacttttaaagttctgctatgtggcgcggtattatcccgtattg
acgccgggcaagagcaactcggtcgccgcatacactattctcagaatgacttggttgagtactcaccagtcacagaaaagcatcttacggat
ggcatgacagtaagagaattatgcagtgctgccataaccatgagtgataacactgcggccaacttacttctgacaacgatcggaggaccga
aggagctaaccgcttttttgcacaacatgggggatcatgtaactcgccttgatcgttgggaaccggagctgaatgaagccataccaaacgac
gagcgtgacaccacgatgcctgtagcaatggcaacaacgttgcgcaaactattaactggcgaactacttactctagcttcccggcaacaatt
aatagactggatggaggcggataaagttgcaggaccacttctgcgctcggcccttccggctggctggtttattgctgataaatctggagccg
gtgagcgtgggtctcgcggtatcattgcagcactggggccagatggtaagccctcccgtatcgtagttatctacacgacggggagtcaggc
aactatggatgaacgaaatagacagatcgctgagataggtgcctcactgattaagcattggtaactgtcagaccaagtttactcatatatacttt
agattgatttaaaacttcatttttaatttaaaaggatctaggtgaagatcctttttgataatctcatgaccaaaatcccttaacgtgagttttcgttc
cactgagcgtcagaccccgtagaaaagatcaaaggatcttcttgagatcctttttttctgcgcgtaatctgctgcttgcaaacaaaaaaaccaccg
ctaccagcggtggtttgtttgccggatcaagagctaccaactctttttccgaaggtaactggcttcagcagagcgcagataccaaatactgttc
ttctagtgtagccgtagttaggccaccacttcaagaactctgtagcaccgcctacatacctcgctctgctaatcctgttaccagtggctgctgcc
agtggcgataagtcgtgtcttaccgggttggactcaagacgatagttaccggataaggcgcagcggtcgggctgaacggggggttcgtgc
acacagcccagcttggagcgaacgacctacaccgaactgagatacctacagcgtgagctatgagaaagcgccacgcttcccgaaggga
gaaaggcggacaggtatccggtaagcggcagggtcggaacaggagagcgcacgagggagcttccagggggaaacgcctggtatcttt
atagtcctgtcgggtttcgccacctctgacttgagcgtcgatttttgtgatgctcgtcaggggggcggagcctatggaaaaacgccagcaac
gcggcctttttacggttcctggccttttgctggccttttgctcacatgt

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A recombinant viral vector comprising:
a transcription termination sequence adjacent to a first inverted terminal repeat (ITR) sequence and/or a transcription termination sequence adjacent to a second inverted terminal repeat (ITR) sequence; and
an expression cassette flanked by at least one of the first and second ITR sequences.

2. The recombinant viral vector of claim 1, wherein the recombinant viral vector comprises a first transcription termination sequence downstream of a first inverted terminal repeat (ITR) sequence and upstream of the expression cassette.

3. The recombinant viral vector of claim 2, further comprising a second inverted terminal repeat (ITR) sequence downstream of the expression cassette, wherein a second transcription termination sequence is optionally downstream of the second ITR sequence.

4. The recombinant viral vector of claim 1, wherein the first inverted terminal repeat sequence and/or the second inverted terminal repeat sequence are wild-type sequences or engineered or variant ITR sequences comprising one or more nucleotide substitutions, additions, or deletions compared to a wild-type sequence.

5. The recombinant viral vector of claim 1, further comprising a polyadenylation (Poly(A)) signal sequence downstream of the expression cassette.

6. The recombinant viral vector of claim 1, wherein the viral vector comprises from 5′ to 3′:
a first inverted terminal repeat sequence, a transcription termination sequence, an expression cassette, and a second inverted terminal repeat sequence; or
a first inverted terminal repeat sequence, a transcription termination sequence, an expression cassette, a Poly(A) signal sequence, and a second inverted terminal repeat sequence.

7. The recombinant viral vector of claim 1, wherein the transcription termination sequence comprises one or more copies of a Poly(A) signal sequence.

8. The recombinant viral vector of claim 1, wherein the expression cassette comprises one or more regulatory control elements and encodes one or more gene products of interest operably coupled to the one or more regulatory control elements.

9. The recombinant viral vector of claim 1, wherein the recombinant viral vector comprises a single strand of DNA.

10. The recombinant viral vector of claim 1, wherein the recombinant viral vector is derived from a virus in the Parvoviridae family, preferably derived from an adeno-associated virus.

11. A virus or virus-like particle comprising the recombinant viral vector of claim 1.

12. A method of delivering a nucleic acid to a cell comprising contacting the cell with an effective amount of a recombinant viral vector of claim 1, or a virus or virus-like particle or composition comprising thereof, wherein the recombinant viral vector comprises the nucleic acid.

13. The method of claim 12, wherein the expression cassette encodes: a system for genetic engineering; a therapeutic or prophylactic gene product; a sequence having homology with a genomic nucleic acid in the cell; or a combination thereof.

14. The method of claim 13, wherein the system for genetic engineering encodes at least one components of a CRISPR-Cas system.

15. The method of claim 12, wherein the cell is in a subject and the method comprises administering the recombinant viral vector, the virus or virus-like particle, or the composition to the subject.
